(19) United States
(12) Patent Application Publication
(10) Pub. No.: US 2014/0044742 A1
(43) Pub. Date: Feb. 13, 2014

(54) METHOD FOR PREPARING A DEPLETED PLASMA MATERIAL CONSISTING OF ONE OR MORE THROMBOGENIC FACTORS

(71) Applicants: Monique Ollivier, Le Kremlin Bicetre (FR); Philippe Paolantonacci, Gif Sur Yvette (FR)

(72) Inventors: Monique Ollivier, Le Kremlin Bicetre (FR); Philippe Paolantonacci, Gif Sur Yvette (FR)

(73) Assignee: LABORATOIRE FRANCAIS DU FRACTIONNEMENT ET DES BIOTECHNOLOGIES, Les Ulis (FR)

(21) Appl. No.: 14/112,181

(22) PCT Filed: Apr. 19, 2012
(86) PCT No.: PCT/EP2012/057228

§ 371 Date: Oct. 16, 2013

(30) Foreign Application Priority Data
Apr. 20, 2011 (FR) 115438

(51) Int. Cl. C07K 1/36

(52) U.S. Cl. CPC C07K 1/36
USPC 424/177.1; 530/390.5

(57) ABSTRACT

The invention concerns a method for preparing a plasma product depleted of one or more thrombogenic factors, comprising the combination of at least two steps chosen from among an ethanol fractionation step, a filtration-adsorption step, a precipitation step with caprylic acid and a chromatography step on ion exchange resin.

### TECHNICAL FIELD

The present invention concerns a method for preparing a plasma product depleted of one or more thrombogenic factors. The present invention also concerns such plasma products obtained using the said method.

### STATE OF THE ART

Human plasma comprises a large quantity of compounds which may be of therapeutic and/or prophylactic interest such as coagulation factors, immunoglobulins or albumin for example. This is why numerous methods for purifying plasma products have been developed, implementing extremely varied techniques.

Among the techniques most used, ethanol fractionation allows the selective separation of the different constituents of plasma, causing these to precipitate under the combined action of ethanol and a low temperature (see Cohn et al. 1946, J. AM. Chem. Soc. 68, 459). The increase in therapeutic standards has nevertheless necessitated the implementation of additional purification steps intended to remove the contaminants likely to trigger anaphylactic reactions or reactions which may be deleterious or pathogenic for the organism of the recipient patients. Among the techniques employed for performing these additional purification steps of plasma products, the techniques of precipitation and chromatographic separation are the most used.

It is thus for example that the implementation of a technique for precipitating proteins in the presence of caprylic acid (also called octanoic acid) allows the removal of most proteins from plasma with the exception of immunoglobulins (see Steinbuch et al., Rev. Franç. Et. Clin. Et Biol. 1969, XIV, 1054).

Various methods have also been developed to increase the purity of the products by implementing chromatographic techniques. Particular mention can be made of patent applications EP 0 703 922 and WO 99/64462, which describe the association of at least two successive chromatography steps, one using anion exchange, the other using cation exchange. The specificity of these methods is provided by the property of the anion exchangers whereby they do not retain immunoglobulin G, under conventional implementation conditions, but fix most of the other proteins co-purified during the pre-purification steps.

Patent application WO 02/092632 filed by the Applicant, discloses a method for preparing concentrates of human immunoglobulin from plasma, comprising an ethanol fractionation step, a precipitation step of the lipid and protein contaminants by means of caprylic acid, tricalcium phosphate or bentonite, and a chromatographic separation on an anion exchanger of alkaline pH, during which the immunoglobulins are adsorbed on the chromatographic support. The method described in application WO 02/092632 may also comprise a viral inactivation step and a concentration step, filtration and lyophilisation of the immunoglobulins concentrate obtained.

Application WO 2007/077365 filed by the Applicant, also describes a method for preparing a concentrate of immunoglobulins G depleted of anti-A and anti-B antibodies, comprising an affinity chromatography step conducted on a support with oligosaccharides having antigenic similarity with the A and B blood groups, and comprising a viral removal step by filtration. The IgG concentrates obtained with the method described in application WO 2007/077365 have a content of anti-A and anti-B antibodies conforming to negative results with the Coombs indirect in vitro test and a low polyreactivity generated by the preparation method, thereby reducing the risks of haemolysis and adverse effects resulting from these antibodies or this polyreactivity.

The subject of the present invention concerns plasma products such as specific immunoglobulins, polyvalent immunoglobulins, enzymes, anaesthesia and intensive care proteins e.g. albumin, fibrinogen or antithrombin.

Known purified plasma products still remain likely to generate adverse effects in patients during therapeutic administering thereof, in particular on account of the fact that they cannot in principle be considered to be fully free of thrombogenic factors. The presence of these thrombogenic factors FXII, FX, FIX, FVII, FVIII and/or FXI and of their activated forms is likely to lead to the formation of thrombosis when the plasma product under consideration is given to patients and could in some extreme cases even lead to patient death. The methods known in the state of the art do not specifically disclose a method for removing these thrombogenic factors in satisfactory manner, insofar as the physicochemical properties thereof and of their activated forms may be extremely close to those of plasma products having a therapeutic interest such as immunoglobulins for example.

There is therefore a strong need for a purification method which allows the preparation of plasma products from human plasma, in which the end quantity of thrombogenic factors such as factors FXI, FXII, FX, FIX, FVII and/or FVIII and in particular FXI, and their activated versions, is null or so low that it does not carry any risk for the health of patients treated with the purified plasma product.

### SUMMARY OF THE INVENTION

The Applicant has developed a method which advantageously allows to prepare a plasma product that is depleted of one or more thrombogenic factors.

By thrombogenic factors is meant factors likely to lead to the formation of thrombosis when they are present as contaminants in a plasma product. Thrombogenic factors may in particular be coagulation factors. In particular, factors FVII, FVIII, FIX, FX, FXI and FXII and their activated forms are thrombogenic factors.

In one preferred embodiment, the plasma product obtained with the method of the invention is depleted of one or more thrombogenic factors. In particular, the plasma product is depleted of FVII, FVIII, FIX, FX, FXI and/or FXII and their activated forms. In particular, the plasma product obtained with the method of the invention is depleted of FVII, FX, FXII and FXI and their activated forms. More particularly it is depleted of FVII and its activated form FVIIa. More particularly, it is depleted of FX and its activated form FXa. More particularly, it is depleted of FXI and its activated form FXIa. More particularly, it is depleted of FXII and its activated form FXIIa.

The present invention is based on the surprising finding that the implementing of a combination of at least two steps chosen from among an ethanol fractionation, adsorption-filtration on a depth filtration filter, a precipitation with caprylic acid and an ion exchange resin chromatography advantageously allows the plasma product to be depleted of one or more thrombogenic factors and their activated forms.

The present invention also concerns a method for preparing a plasma product depleted of thrombogenic factors, comprising the combination of at least two steps, preferably at least three steps, chosen from among:
an ethanol fractionation step;
a filtration-adsorption step;
a precipitation step with caprylic acid; and
a chromatography step on ion exchange resin.

In one particular embodiment, the method for preparing a plasma product depleted of thrombogenic factors of the invention comprises the successive steps of:
an ethanol fractionation step; and
a filtration-adsorption step.

In one particular embodiment, the method for preparing a plasma product depleted of thrombogenic factors of the invention comprises the successive steps of:
an ethanol fractionation step; and
a precipitation step with caprylic acid.

In one particular embodiment, the method for preparing a plasma product depleted of thrombogenic factors of the invention comprises the successive steps of:
an ethanol fractionation step; and
a chromatography step on ion exchange resin.

In one particular embodiment, the method for preparing a plasma product depleted of thrombogenic factors of the invention comprises the successive steps of:
a filtration-adsorption step; and
an ethanol fractionation step.

In one particular embodiment, the method for preparing a plasma product depleted of thrombogenic factors of the invention comprises the successive steps of:
a filtration-adsorption step; and
a precipitation step with caprylic acid.

In one particular embodiment, the method for preparing a plasma product depleted of thrombogenic factors of the invention comprises the successive steps of:
a filtration-adsorption step; and
a chromatography step on ion exchange resin.

In one particular embodiment, the method for preparing a plasma product depleted of thrombogenic factors of the invention comprises the successive steps of:
a precipitation step with caprylic acid; and
a chromatography step on ion exchange resin.

In one particular embodiment, the method for preparing a plasma product depleted of thrombogenic factors of the invention comprises the successive steps of:
an ethanol fractionation step;
a filtration-adsorption step; and
a precipitation step with caprylic acid.

In one particular embodiment, the method for preparing a plasma product depleted of thrombogenic factors of the invention comprises the successive steps of:
a filtration-adsorption step;
an ethanol fractionation step; and
a precipitation step with caprylic acid.

In one particular embodiment, the method for preparing a plasma product depleted of thrombogenic factors of the invention comprises the successive steps of:
an ethanol fractionation step;
a precipitation step with caprylic acid; and
a chromatography step on ion exchange resin.

In one particular embodiment, the method for preparing a plasma product depleted of thrombogenic factors of the invention comprises the successive steps of:
an ethanol fractionation step;
a filtration-adsorption step; and
a chromatography step on ion exchange resin.

Advantageously, the preparation method of the present invention further comprises one or more chromatography steps, preferably one or more chromatography steps on ion exchange resin.

In one particular embodiment, the method for preparing a plasma product depleted of thrombogenic factors of the invention comprises the successive steps of:
a filtration-adsorption step;
a precipitation step with caprylic acid; and
a chromatography step on ion exchange resin.

In one particular embodiment, the method for preparing a product depleted of thrombogenic factors of the invention comprises the successive steps of:
an ethanol fractionation step;
a filtration-adsorption step;
a precipitation step with caprylic acid; and
a chromatography step on ion exchange resin;

or consisting of:
a filtration-adsorption step;
an ethanol fractionation step;
a precipitation step with caprylic acid; and
a chromatography step on ion exchange resin.

In one particular embodiment, the filtration-adsorption step is conducted on a depth filtration filter comprising cellulose and perlites, and a charged resin. Preferably, the filter used for the filtration-adsorption step has a grade ranging from 0.1 to 0.4 μm, preferably from 0.2 to 0.4 μm, more preferably from 0.25 to 0.35 μm, preferably of 0.3 μm.

In one particular embodiment, the preparation method of the present invention comprises one or two additional steps chosen from among the steps of viral inactivation, preferably by solvent-detergent, and viral removal preferably by nanofiltration.

In one particular embodiment, the preparation method of the present invention comprises one or two additional steps chosen from among the steps of viral inactivation, preferably by solvent-detergent, and affinity chromatography on a support comprising oligosaccharides having antigenic similarity with the blood groups A and B.

In one particular embodiment, the preparation method of the present invention comprises a final step of concentration, filtration and the addition of a pharmaceutically acceptable stabilizer, then of packaging as a sterile solution and optionally freezing and lyophilisation.

Other characteristics and advantages of the invention will become apparent on reading the following description of a preferred embodiment of the invention given as an example.

### DETAILED DESCRIPTION OF EMBODIMENTS OF THE INVENTION

In the present invention, by <<plasma product>> is meant any compound, any protein, such as an immunoglobulin or albumin, fibrinogen or antithrombin or any mixture of proteins able to be purified from human plasma. For example, the product may be specific immunoglobulin, polyvalent immunoglobulin, enzymes, proteins for anaesthesia or intensive care such as albumin, fibrinogen or antithrombin. The plasma product of the invention preferably corresponds to an immunoglobulin G (or IgG), an immunoglobulin A (or IgA), an immunoglobulin M (or IgM), an immunoglobulin E (or IgE) or a mixture of one or more of these, and is under a solution or a concentrate form. In one particular embodiment, the plasma product of the invention corresponds to a concentrate of immunoglobulins specifically directed against a particular antigen, such as anti-rhesus or anti-D cytotoxic antibodies for example. In another embodiment, the plasma product of the invention may also correspond to a mixture of polyvalent immunoglobulins, composed of 97% IgG and directed against a wide diversity of antigens. In another embodiment, the plasma product of the invention may also correspond to albumin, fibrinogen or antithrombin.

By <<products obtained according to the method of the invention>> is meant any product of plasma product type obtained directly or indirectly with the method of the invention or able to be obtained using this method.

In general, when reference is made to a thrombogenic factor, reference is made to the inactivated or activated forms of these factors. Indeed, the thrombogenic effects of these factors are particularly marked when they are in activated form. The method of the present invention surprisingly allows the removal of thrombogenic factors in particular when they are in activated form. In particular, when reference is made to <<factor XI>> or <<FXI>> in the present application, it is therefore meant factor XI in its inactive form and factor XI in its activated form (also denoted FXIa). The same applies to each of the other thrombogenic factors cited in the present application.

By <<ethanol fraction>> in the meaning of the present invention is meant plasma fractions obtained at different steps of ethanol fractionation of plasma such as known to persons skilled in the art.

Under the present invention, by <<depleted of thrombogenic factors>> is meant that the plasma product obtained according to the method of the invention no longer contains any thrombogenic factor(s), or only contains non-significant traces thereof. As a result, the plasma product of the invention cannot therefore lead to the formation of thrombosis in a patient caused by the triggering of coagulation in the presence of contaminating thrombogenic factor(s).

By <<depleted of FVII, of FVIII, of FIX, or FX and/or of FXII>> it is therefore meant that the plasma product obtained according to the method of the invention no longer contains FVII, FVIII, FIX, FX and/or FXII or that it only contains residual traces thereof. As a result, the plasma product obtained with the method of the invention cannot therefore lead to the formation of thrombosis caused by the triggering of coagulation in the presence of any one of the factors FVII, FVIII, FIX, FX and/or FXII.

In one preferred embodiment, the plasma product obtained according to the method of the invention is depleted of FVII and/or depleted of FX.

In one preferred embodiment, the plasma product obtained according to the method of the invention is depleted of FXI. By <<plasma product depleted of FXI>> it is meant a plasma product, in particular a plasma product of polyvalent immunoglobulins type, having less than 0.8 mU/mg Ig, preferably less than 0.5 mU/mg Ig of FXI.

In another preferred embodiment, the plasma product obtained according to the method of the invention is depleted of FXII. By <<plasma product depleted of FXII>> it is meant a plasma product, in particular a plasma product of polyvalent immunoglobulins type, having less than 20 μU of FXII/mg Ig, preferably less than 10 μU of FXII/mg Ig.

In one particular embodiment of the present invention, the plasma product depleted of one or more thrombogenic factors can be in liquid or lyophilised form, and/or be packaged in the presence of suitable stabilizers. It can also be stored until subsequent use. The added stabilizers advantageously allow the ensured stability of the plasma product over time and make lyophilisation possible by avoiding denaturing of the plasma product during lyophilisation and during storage. The stabilizers used advantageously correspond to those described by the Applicant in its patent application WO 2004/091656, namely a mixture of sugar alcohol, glycine and a non-ionic detergent.

The purified plasma products of the invention are intended for therapeutic use and can be the subject of administration via systemic, oral, parenteral, intravenous, intramuscular, per or transcutaneous, nasal, rectal, perlingual, ocular or respiratory route. For this purpose, the plasma products of the invention depleted of one or more thrombogenic factors are virally safe, for example by using a conventional viral inactivation treatment (for example solvent-detergent treatment) and/or viral removal treatment (for example by nanofiltration).

In one preferred embodiment, when the plasma product to be purified corresponds to immunoglobulins, chromatographic separation is conducted on a resin of anion exchange type, preferably on a resin of type Diethylaminoethyl (DEAE), Trimethylaminoethyl (TMAE) or quaternary amine (QAE).

In one particular embodiment of the present invention, the method of the invention may comprise additional purification steps such as affinity chromatographies or hydrophobic chromatographies.

If the purified plasma product of the invention corresponds to immunoglobulins, the method of the present invention comprises an additional separation step of affinity chromatography on a chromatography support comprising oligosaccharides having antigenic similarity with the blood groups A and B. This additional purification step advantageously allows the removal of the anti-A and anti-B antibodies of the purified plasma product obtained at the preceding step. This removal can be performed following the method described in patent application WO 2007/077365.

In one particular embodiment, the method of the invention also comprises at least one inactivation and/or removal step of infectious agents such as viruses or prions. Viral inactivation is preferably performed by heat treatment or by chemical treatment such as treatment of solvent-detergent type. Solvent-detergent treatment can be implemented using for example a mixture of tri-n-butylphosphate (TnBP) and a detergent chosen from among Triton X-100 or Tween 80. It is also possible to conduct viral inactivation by subjecting the purified plasma product to UV or gamma ray radiation. Viral removal is preferably performed by nanofiltration on nanometre filters having decreasing porosity ranging from 100 nm to 15 nm. The nanofiltration step advantageously allows the removal of possible pathogens (viruses or prions for example) which may not have been removed by the solvent-detergent treatment. Viral removal can be carried out on any suitable filter, preferably on filter Planova 35 nm, Planova 20 nm and/or Planova 15 nm filters. The nanofiltration can, when applicable, be carried out after optional concentration and/or ultrafiltration steps of the purified plasma product.

In one particular embodiment, the method of the present invention may also comprise a concentration and/or ultrafiltration step of the purified plasma product. The purified plasma product may also be subjected to a sterilizing filtration step and/or a freezing and lyophilisation step. It can also be stored until subsequent use. The added stabilizers advantageously allow the ensured stability of the plasma product over time and make lyophilisation possible whilst avoiding denaturation of the plasma product during this process and when in storage. The stabilizers used advantageously correspond to those described by the Applicant in its patent application WO 2004/091656, namely a mixture of sugar alcohol, glycine and a non-ionic detergent.

In one preferred embodiment, the method of the present invention comprises the successive implementation of:
an ethanol fractionation step;
a filtration-adsorption step; and
a precipitation step with caprylic acid. or:
a filtration-adsorption step;
an ethanol fractionation step; and
a precipitation step with caprylic acid.

These combinations of steps advantageously allow the removal of thrombogenic factors present in the starting plasma product, and in particular Factors FVII, FVIII, FIX, FX, FXI and/or FXII.

These combinations of steps, of comparable efficacy, more particularly allow the removal of Factor FXI, FVII, FX and/or FXII down to a factor concentration lower than their respective detection threshold.

Evidently, the present invention is not limited to the examples and to the embodiments described and illustrated, but may be given numerous variants accessible to those skilled in the art.

The following examples illustrate the invention without, however, limiting the scope thereof.

### EXAMPLES

### Example 1

### Removal of FXI on Filter

The different filtration steps are conducted at a temperature of between 4° C. and 10° C. The charged filters are previously rinsed with a minimum of 6 mL of equilibration buffer solution (CTS 2.94 g/L, Na2HPO4 1.79 g/L, KH2PO4 0.7 g/L, NaCl 3.5 g/L) per cm2 of filtering surface by applying a flow rate of 7.5 mL/h/cm2. A variable volume of cryoprecipitation supernatant is filtered. The flow rate is measured by weighing the filtered fraction at regular time intervals. The measured flow rate after the first 5 minutes of filtration is considered as the initial flow rate. Depending on the tests, the filter is rinsed after injecting the raw material with a variable volume of equilibration buffer solution allowing examination of the influence of filter rinsing on the retention of factor XI and on the recovery of non-adsorbed proteins. To test the retention efficacy of filtration-adsorption, the FXI is eluted with the elution buffer solution. Samples of the homogenized starting fractions, filtrate and eluate were taken, then aliquoted, identified and stored at a temperature equal to or lower than −70° C.

The results obtained throughout the filtration tests for the tested filter are given in the Table below and show the FXI-retention capacity of a depth filtration filter comprising cellulose and perlites, and a charged resin, e.g. the Sartoclear® P 24 cm2 filter, the said filter having a grade ranging from 0.1 to 0.4 μm.

| | | | | FXI | |
|---|---|---|---|---|---|
| | | Flow | | retention | Collected |
| | | rate | FXI loss | yield | filtrate |
| Tested | Load | (mL/h/ | in the | (Eluate 1M | volume |
| filter | (ml/cm2) | cm2) | filtrate | NaCl) | (mL/cm2) |
| Sartoclear ® | 4.2 | 5 | <detection | 99% | 7.9 |
| P 24 cm2 | | | threshold | | |
| Sartoclear ® | 14.6 | 5 | <detection | 72% | 21.5 |
| P 24 cm2 | | | threshold | | |

### Example 2

### Removal of FXI by Chromatography

The different filtration steps are conducted at a temperature of between 4° C. and 10° C.

After clarification by passing through 0.22 μm Millipak, the cryoprecipitation supernatant is injected into a packed column with the tested gel equilibrated with the equilibration buffer solution (CTS 2.94 g/L, Na2HPO4 1.79 g/L, KH2PO4 0.7 g/L, NaCl 3.5 g/L). The gel is then washed with the same buffer solution until return to baseline.

To test the retention efficacy of chromatography, the FXI is then eluted by passing elution buffer solution of very high ionic strength without the gel pre-washing step to remove the proteins weakly attached to the gel.

Samples are taken from the collected, homogenized fractions, aliquoted, identified and stored at a temperature equal to or lower than −70° C. until biochemical assay.

The results obtained for chromatography testing with different chromatography columns are given in the table below.

| | Load FXI | Flow | | |
|---|---|---|---|---|
| | applied | rate | FXI loss | FXI |
| | (U/mL | (mL/h/ | in the | retention |
| Test conditions | gel) | cm2) | filtrate | yield |
| SP Sepharose | 12 | 150 | <21% | 92.5% |
| [symbol] Big Beads [symbol] | | | | |
| K15 Column packed with | | | | |
| gel to height of 5 cm | | | | |
| 7° C., 1M eluate | | | | |
| Streamline SP in fixed | 40 | 60 | 23.6% | 78.3% |
| bed, K26 column (5 cm2) | | | | |
| packed with gel to | | | | |
| height of 1 cm 12° C. | | | | |
| 1M Eluate | | | | |
| Streamline SP in | 63 | 300 | 28.1% | 70.5% |
| fluidized bed K26 Column | | | | |
| (2 cm2) packed with gel | | | | |
| to height of 5.5 cm | | | | |
| 5.5° C. Eluate 1M | | | | |

### Example 3

### A: Ethanol Fractionation, Caprylic Precipitation and Chromatography

As starting material 1 kg of ethanol precipitate is used “fraction I+fraction II+fraction III” obtained from plasma according to the Cohn method or the Kistler and Nitschmann's method (1962, Vox Sang, 7,414). This precipitate is re-suspended in acetate buffer (sodium acetate-acetic acid) at a pH 4.7-4.9 under agitation at 20° C.

Caprylic acid is added to the re-suspended precipitate. The addition must be made slowly at ambient temperature. To the mixture is added a filtration adjuvant and the precipitate is separated by filtration using a press filter.

The filtrate is recovered, clarified and concentrated by ultrafiltration then it is subjected to sterile filtration at 0.45 μm and 0.2 μm. It is then subjected to viral inactivation treatment using solvent/detergent as described by Neurath and Horowitz (patent U.S. Pat. No. 4,764,369). A mixture of Triton X100/TnBP is used.

The mixture is adjusted to 64 g/l of proteins at pH 6.5. Contact is maintained for 4 to 6 hours at between 4 and 25° C. The pH is then adjusted to 9 with NaOH. The mixture is then subjected to an anion exchange chromatography using a column. As anion exchange material TMAE Fractogel® is used, loaded in a chromatography column, equilibrated with glycine-NaCl buffer (glycine 0.676 g/l-NaCl 0.526 g/l) at pH 9. The mixture is loaded into the column in a proportion of 50 g of proteins per 1 litre of gel. After loading, the column is washed with glycine-NaCl buffer, pH 9 (the same as for equilibration). Washing is monitored by the optical density of the effluent at 280 nm. After return to baseline, the column is eluted with phosphate buffer at pH 6.2 (disodium hydrogenophosphate-sodium dihydrogenophosphate). The eluate containing immunoglobulins G is adjusted to pH 4.5 and subjected to ultrafiltration using cassettes.

The solution is then subjected to sterile filtration at 0.22 μm followed by nanometric filtration using three filters arranged in series and with decreasing retention thresholds of 100, 50 and 20 nanometres. Filtration was followed by ultrafiltration using cassettes to obtain a concentration of the final solution of 120-150 g/l.

### B: IgIV Comparison Regarding Factor XI and Factor XII Levels

A concentrate of immunoglobulins prepared according to the method described in Example 3A is compared with other concentrates of immunoglobulins on the market.

A concentrate of IgIV prepared according to the method described in Example 3A has a factor XI content of less than 0.5 mU/mg Ig whereas the other concentrates of immunoglobulins on the market have factor XI levels of more than 0.8 mU/mg Ig and some have a factor XI content of more than 2 mU/mg Ig.

The level of factor XII is also evaluated. The immunoglobulin concentrates on the market have a factor XII content of more than 20 μU/mg Ig, some having a factor XII content of more than 30 μU/mg Ig, whereas a IgIV concentrate prepared according to the method described in Example 1 has a factor XII content of less than 10 μU/mg Ig.

### C: Assay of Factors VII and X Before and After the Method Comprising the Steps of Ethanol Fractionation, Caprylic Precipitation and Chromatography.

The contents of Factors VII and X are measured before and after the method such as described in Example 3A.

The contents in the plasma (before the method comprising ethanol fractionation, caprylic precipitation and chromatography) correspond to the mean assayed values for six 4500 litre plasma batches.

| Factor | | VII | X |
|---|---|---|---|
| Plasma | Volume (I) | 4500 | 4500 |
| | CC (EUI/ml) | 0.66 | 0.39 |
| | Total (EUI) | 2970000 | 1755000 |
| P1 - after ethanol | Volume (I) | 431 | 431 |
| fractionation, caprylic | CC (mEUI/ml) | (<)3.1 | (<)0.7 |
| precipitation and | Total (EUI) | 1336.1 | 301.7 |
| chromatography | | | |

| | | |
|---|---|---|
| Removal factor (P1/Plasma) | 2223 | 5817 |
| Log | (>)3.3 | (>)3.8 |

CC: concentration - (<): concentration lower than detection threshold; in this case the detection threshold is used for calculations

After the steps of ethanol fractionation, caprylic precipitation and chromatography, the content of FVII antigen is lower than the detection limit.

The P1/plasma ratio shows a decrease of more than 3 logs related to the method comprising the steps of ethanol fractionation, caprylic precipitation and chromatography.

As for Factor VII, after the steps of ethanol fractionation, caprylic precipitation and chromatography, the content of FX antigen is lower than the detection limit.

The P1/plasma ratio shows a decrease of close to 4 logs related to the method comprising the steps of ethanol fractionation, caprylic precipitation and chromatography.

Example 3C clearly shows that a method comprising the steps of ethanol fractionation, caprylic precipitation and chromatography according to the invention allows the obtention of a plasma product depleted of thrombogenic factors.

### Example 4

### A: Filtration, Ethanol Fractionation and Caprylic Precipitation

Cryoprecipitation supernatant is filtered on a depth filtration filter comprising cellulose and perlites and a charged resin e.g. the Sartoclear® P 24 cm2 filter, the grade of the said filter ranging from 0.1 to 0.4 μm.

The filtrate is subjected to ethanol fractionation following the method of Cohn or Kistler and Nitschmann (1962, Vox Sang. 7,414). The ethanol precipitate “fraction I+fraction II+fraction III” is re-suspended in acetate buffer (sodium acetate-acetic acid) at pH 4.7-4.9, under agitation, at 20° C.

Caprylic acid is added to the re-suspended precipitate. The addition must be made slowly at ambient temperature. To the mixture is added a filtration adjuvant and the precipitate is separated by filtration using a press filter.

The filtrate is recovered, clarified and concentrated by ultrafiltration, then it is subjected to sterile filtration at 0.45 μm and 0.2 μm.

### B: Assay of Factors VII and X Before and After the Method Comprising the Steps of Filtration, Ethanol Fractionation and Caprylic Precipitation.

The contents of Factors VII and X are measured before and after the method such as described in Example 4A.

The contents before the method correspond to the mean assayed values for six 4500 litre plasma batches.

| Factor | | VII | X |
|---|---|---|---|
| Plasma | Volume (I) | 4500 | 4500 |
| | CC (EUI/ml) | 0.85 | 0.75 |
| | Total (EUI) | 3825000 | 3375000 |
| P1 - after filtration, | Volume (I) | 436 | 436 |
| ethanol fractionation, and | CC (mEUI/ml) | (<)3.1 | (<)0.7 |
| caprylic precipitation | Total (EUI) | 1351.6 | 305.2 |

| | | |
|---|---|---|
| Removal factor (P1/Plasma) | 2830 | 11058 |
| Log | (>)3.5 | (>)4.1 |

CC: concentration - (<): concentration lower than the detection threshold; in this case, the detection threshold is used for calculations.

After the steps of filtration, ethanol fractionation and caprylic precipitation, the content of FVII antigen is lower than the detection limit.

The P1/plasma ratio shows a decrease of more than 3 logs related to the method comprising the steps of filtration, ethanol fractionation and caprylic precipitation.

As for Factor VII, after the steps of ethanol fractionation, caprylic precipitation and chromatography, the content of F X antigen is lower than the detection limit.

The P1/plasma ratio shows a decrease of more than 4 logs related to the method comprising the steps of filtration, ethanol fractionation and caprylic precipitation.

Example 4B clearly shows that a method comprising the steps of filtration, ethanol fractionation and caprylic precipitation according to the invention allows the obtention of a plasma product depleted of thrombogenic factors.

1-16. (canceled)

17. A method for preparing a plasma product depleted of one or more thrombogenic factors, comprising at least two steps selected from the group consisting of:
ethanol fractionation;
filtration-adsorption;
precipitation with caprylic acid; and
chromatography on an ion exchange resin.

18. The method according to claim 17, comprising successive steps of:
ethanol fractionation; and
precipitation with caprylic acid.

19. The method according to claim 17 comprising successive steps of:
filtration-adsorption; and
ethanol fractionation.

20. The method according to claim 17 comprising successive steps of:
precipitation with caprylic acid; and
chromatography on an ion exchange resin.

21. The method according to claim 17, comprising successive steps of:
ethanol fraction;
precipitation with caprylic acid; and
chromatography on an ion exchange resin.

22. The method according to claim 17, comprising successive steps of:
filtration-adsorption;
ethanol fractionation; and
precipitation with caprylic acid.

23. The method according to claim 17, comprising successive steps of:
ethanol fractionation;
filtration-adsorption;
precipitation with caprylic acid.

24. The method according to claim 22, further comprising a step of chromatography on and ion exchange resin.

25. The method according to claim 17, wherein the filtration-adsorption step is conducted on a depth filtration filter comprising cellulose and perlites, and a charged resin, wherein the filter has a grade ranging from 0.1 to 0.4 μm.

26. The method according to claim 25, wherein the filter has a grade ranging from 0.2 to 0.4 μm.

27. The method according to claim 26, wherein the filter has a grade ranging from 0.25 to 0.35 μm.

28. The method according to claim 27, wherein the filter has a grade of 0.3 μm.

29. The method according to claim 17, further comprising an additional viral inactivation step and/or an additional viral removal step.

30. The method according to claim 29, wherein the viral inactivation step comprises use of solvent-detergent and the additional viral removal step comprises nanofiltration.

31. The method according to claim 17, further comprising an additional affinity chromatography step on a support comprising oligosaccharides having antigenic similarity with blood groups A and B.

32. A plasma product depleted of one or more thrombogenic factors obtainable by the method according to claim 17.

33. The plasma product according to claim 32, wherein the plasma product is depleted of thrombogenic factor FXI, of thrombogenic factor FVII, of thrombogenic factor FX and/or of thrombogenic factor FXII.

34. The plasma product according to claim 33, wherein the plasma product is an immunoglobulin solution.

35. The plasma product according to claim 32, wherein the plasma product has a concentration of FXI equal to or less than 0.8 mU/mg Ig.

36. The plasma product according to claim 35, wherein the concentration of FXI is equal to or less than 0.5 mU/mg Ig.

37. A pharmaceutical product, comprising the plasma product according to claim 32 and at least one pharmaceutically acceptable vehicle.
